(19) United States
(12) Patent Application Publication
(10) Pub. No.: US 2008/0171123 A1
(43) Pub. Date: Jul. 17, 2008

(54) Technically Produced Dihydrocoumarin

(76) Inventors: Hans Henning Wenk, Essen (DE); Wilfried Schwab, Marktheidenfeld (DE); Katrin Haser, Althegnenberg (DE)

Correspondence Address: FULBRIGHT & JAWORSKI, LLP, 666 FIFTH AVE, NEW YORK, NY, 10103-3198, US

(21) Appl. No.: 11/659,595

(22) PCT Filed: Aug. 5, 2005
(86) PCT No.: PCT/EP2005/008512

§ 371 Date: Sep. 19, 2007

(30) Foreign Application Priority Data
Aug. 6, 2004 (DE) 10 2004 038 154.2

(51) Int. Cl. A23L 1/22; C12P 17/06; A21D 13/00; A23L 2/00; A23L 1/10; A23C 9/152; A23C 13/00; A23G 3/48; C07D 311/10

(52) U.S. Cl. 426/536; 435/125; 549/290; 426/549; 426/660; 426/590; 426/586; 426/618; 426/580

(57) ABSTRACT

Dihydrocoumarin produced technologically from coumarin by biotransformation is claimed together with related production variants that are carried out with the aid of isolated enzymes and/or microorganisms. Pure coumarin as well as coumarin isolated from a plant extract or coumarin-containing plant extracts can be used as starting materials. Selected strains of Saccharomyces, Arthrobacter, Pseudomonas, Bacillus, Basidiomycetes and Fusarium can be directly used for the biotransformation or can provide the enzymes required for the biotransformation. Preferred enzymes are coumarate and coumarin reductases. A production variant is additionally claimed which starts with coumarin and which, via o-coumaric acid or via intermediary dihydrocoumarin, leads to the subsequent melilotic acid which is subsequently dehydrated to dihydrocoumarin. The dihydrocoumarin obtained in this manner fulfils all criteria of a natural raw material which is why the product can also be used as a natural flavouring or for the production of or as a component of natural, nature-identical and synthetic flavours as they are typically used in baked goods, sweets, beverages, crèmes, cereal products and milk products.

The present invention concerns dihydrocoumarin that has been produced technologically, a process for the production thereof and its use.

Dihydrocoumarin (CAS: 119-84-6) is a 3,4-dihydro-2H,1-benzopyran-2-one which is contained naturally for example in Melilotus officinalis. Dihydrocoumarin (DHC) is a flavouring which is used in sweet tasting flavour notes such as caramel, vanilla or rum. The scent note of DHC is usually characterized as sweet, woody, grass-like or as a typical caramel and vanilla taste.

Due to its defined structure, DHC can be declared as a natural ingredient where the term “natural flavour” can be used among others for compounds that can be obtained from materials of natural origin by enzymatic or microbiological processes (US 21CFR101.22 (3), EU Directive 88/388/EEC). In line with the steadily increasing demand for untreated or natural foods higher prices can be achieved with natural flavourings compared with synthetic compounds which is why biotechnological production is an interesting alternative to chemical synthesis not only for economic reasons.

In addition to Melilotus officinalis (genuine stone clover), traces of DHC are also naturally present in, for example, extracts of the tonka bean (Dipteryx odorata) (HPLC analysis of tonka bean extracts, D. Ehlers, M. Pfister, W.-R. Bork, P. Toffel-Nadolny, Z. Lebensm. Unters. Forsch. 1991, 193, 21 to 25). However, the amounts of DHC that occur naturally are too small to justify an economically worthwhile extraction of DHC from these natural sources. On the other hand, some plants with mentionable contents of DHC also at the same time contain high amounts of unsaturated coumarin (CAS: 91-64-5) where the tonka bean is again mentioned as an example with coumarin contents of up to 10%. Coumarin is naturally metabolized by certain plants, bacteria and fungi to dihydrocoumarin or also to melilotic acid.

The bacterial metabolism of coumarin typically takes place in such a manner that o-coumaric acid is enzymatically converted in the presence of NADH to melilotic acid by for example Arthrobacter species (The metabolism of coumarin by a microorganism. II. The reduction of o-coumaric acid to melilotic acid, C. C. Levy, G. D. Weinstein, Biochemistry 1964, 3(12), 1944 to 1947).

In fact, no enzymatic hydrolysis whatsoever of dihydrocoumarin to melilotic acid could be detected in the organism in this process using a purified enzyme which acts substrate-specifically with regard to o-coumaric acid. This, on the other hand, allows the conclusion that the reaction of coumarin to melilotic acid occurs in this Arthrobacter organism by a hydrolysis of coumarin to o-coumaric acid and its subsequent reduction to melilotic acid. This assumption was confirmed by investigations in which isotopes that originated from tritium-labelled coumarin and were incorporated with the aid of a purified enzyme, were found in o-coumaric acid and melilotic acid (Metabolism of coumarin by a microorganism: o-coumaric acid as an intermediate between coumarin and melilotic acid. C. C. Levy, Nature 1964, 204(4963), 1059 to 1061). The reduction step of o-coumaric acid to melilotic acid has proven to be irreversible and crude extracts of the enzyme were able to degrade melilotic acid to a compound which had similar properties to 2,3-dihydroxyphenyl-propionic acid.

The direct reduction of coumarin to dihydrocoumarin by enzymes from Pseudomonas species has also been described (The metabolism of coumarin by a strain of Pseudomonas, Y. Nakayama, S. Nonomura, C. Tatsumi, Agr. Biol. Chem. 1973, 37(6), 1423-1437). The microorganism used for this was obtained with the aid of enrichment cultures from soil isolates, wherein coumarin was used as the only source of carbon. It was also possible to isolate a coumarin reductase from a crude cell extract which enabled coumarin to be converted into dihydrocoumarin in the presence of NADH. The enzyme used for this proved to be highly specific, but was not able to catalyse the conversion of o-coumaric acid or various substituted coumarin derivatives. Furthermore, this isolated enzyme was not subject to product inhibition by dihydrocoumarin. The isolation of a melilotate-/o-coumarate-hydroxylase which is able to convert 2-monohydroxy acids into the corresponding 2,3-dihydroxy compounds in the presence of NADH and oxygen was equally successful.

The formation of DHC by an enzymatic Bayer-Villinger oxidation of indanone was also observed for an Arthrobacter strain which was able to form dihydrocoumarin by the fluorene degradation pathway (New metabolites in the degradation of fluorene by Arthrobacter sp. strain F101, M. Casellas, M. Grifoll, J. M. Bayona, A. M. Solanas, I Appl. Environm. Microbiol. 1997, 63(3), 819 to 826). In addition a dihydrocoumarin hydrolase activity was found in these investigations in crude extracts of fluorene-injected cells which is why no accumulation of larger amounts of DHC was observed. On the basis of these findings it was proposed that melilotic acid as a typical hydrolysis product is metabolized by a β-oxidation to salicylic acid. It was possible to accumulate up to 3% by weight DHC in neutral extracts with the aid of incubation experiments with 1-indanone.

Since DHC is also regarded as a potential cell poison, it was no surprise that the irreversible degradation of DHC to melilotate was demonstrated with the aid of an enzyme from Fusarium oxisporum (Purification and characterization of a novel lactonohydrolase, catalyzing the hydrolysis of aldonate lactones and aromatic lactones, from Fusarium oxysporum, S. Shimizu, M. Kataoka, K. Shimizu, M. Hirakata, K. Sakamoto and H. Yamada, Eur. J. Biochem. 1992, 209, 383 to 390).

Similar findings to those of bacteria were also obtained with fungi. Thus, a significant reduction of the coumarin concentration was observed when strains of Aspergillus niger were incubated in the presence of coumarin. Melilotic acid proved to be the main product of the biotransformation while smaller amounts of o-coumaric acid and traces of 4-hydroxycoumarin and catechol were also present (Fungal Metabolism—I. The transformations of coumarin, o-coumaric acid and trans-cinnamic acid by Aspergillus niger. S. M. Bockws, Phytochemistry 1967, 6, 127 to 130).

Shieh et al. succeeded in isolating soil fungi with the aid of enrichment cultures in which coumarin served as the sole source of carbon (Use of Coumarin by soil fungi, H. S. Shieh, A. C. Blackwood, Can. J. Microbiol. 1969, 15(6), 647 to 648). In this enrichment culture Fusarium solani proved to be the most active isolate and melilotic acid was the main product of the coumarin-containing medium inoculated with fusarium. The conversion of coumarin was accelerated by aerating the medium and it was additionally increased by adding traces of iron and manganese. When the culture was grown in a medium containing o-coumaric acid as the sole source of carbon, high yields of 4-hydroxycoumarin were obtained. It was also observed that Fusarium solani grows rapidly on dihydrocoumarin which was almost quantitatively converted into melilotic acid. Since the overall conversion of coumarin to melilotic acid by cell-free extracts of Fusarium solani only proceeded very slowly but the conversion of dihydrocoumarin to melilotic acid was very rapid, the authors concluded that the formation of melilotic acid from coumarin occurs via the intermediate formation of dihydrocoumarin.

T. Kosuge et al. (The metabolism of aromatic compounds in higher plants. I. Coumarin and o-coumaric acid. T. Kosuge, E. E. Conn, J. Biol. Chem. 1959, 234(8), 2133 to 2137) demonstrated that shoots of Melilotus alba are able to convert coumarin to a mixture of melilotic acid and β-glucosides of melilotic and o-coumaric acid. In contrast to the degradation pathway in Arthrobacter but in agreement with the observations with Fusarium solani, the initial reduction step of coumarin to dihydrocoumarin was followed by its hydrolysis to melilotic acid which, however, occurred without the accumulation of large amounts of DHC. Dihydrocoumarin hydrolase was isolated from Melilotus alba as the enzyme that is responsible for the catalysis of the hydrolysis step. An even higher DHC hydrolase activity was reported for Melilotus officinalis (The metabolism of aromatic compounds in higher plants. V. Purification and properties of dihydrocoumarin hydrolase of Melilotus alba. T. Konsuge, E. E. Conn, J. Biol. Chem. 1962, 237(5), 1653 to 1656).

With regard to DHC as a flavouring and aroma substance there are numerous references to synthetic processes not only in the patent literature. However, the synthetic routes described in this literature only comprise chemical reaction steps and only provide artificial DHC or DHC that is identical to natural DHC. Thus, the reduction of coumarin to dihydrocoumarin with the aid of palladium catalysts is described in U.S. Pat. No. 6,462,203.

In contrast, a process which, especially with regard to economic aspects, is suitable for producing a natural dihydrocoumarin is not known from the previous state of the art which is why up to now natural DHC has also not been in circulation as a commercial product.

Hence, this serious disadvantage of the state of the art has given rise to the object of the present invention to provide a new dihydrocoumarin and also a suitable production process for this purpose.

This object was achieved by providing a natural dihydrocoumarin that is produced technologically from coumarin by biotransformation.

It surprisingly turned out that this dihydrocoumarin cannot only be produced with the aid of a completely natural system and thus fulfils the criterion of being a natural substance, but can also be obtained in yields and in a product quality which allow it to be used economically in fields of application that have previously been the exclusive preserve of synthetic variants. In fact, naturally produced DHC has a significantly better product quality than the synthetic variants. The product quality is mainly based upon the absence of byproducts or undesired secondary products such as those that are known to be disadvantageous from the technical reaction.

In addition to dihydrocoumarin itself produced technologically by biotransformation from coumarin, the present invention also encompasses a process for its production in which the biotransformation is carried out in particular with the aid of isolated enzymes and/or microorganisms. Pure coumarin or coumarin from a plant extract is a preferred starting material for the present invention. In this connection, the coumarin-containing plant extracts which are preferably not additionally purified, additionally concentrated or otherwise specially treated, can also be used as such.

Coumarate reductases (EC 1.3.1.11) or coumarin reductases which are derived in particular from Melilotus species such as Melilotus officinalis or Melilotus alba are recommended as suitable enzymes for the enzymatic biotransformation. Particularly suitable enzymes from microorganisms are those from Saccharomyces, Arthrobacter, Pseudomonas, Bacillus, Basidiomycetes and Fusarium in which case the presence of cofactors is recommended where appropriate.

If the biotransformation should not be carried out with isolated enzymes, but rather with microorganisms, the invention envisages that this bioconversion is carried out with representatives of microorganisms from which the enzymes that are used alternatively are usually derived, i.e. Saccharomyces, Arthrobacter, Pseudomonas, Bacillus, Basidiomycetes and Fusarium.

As already mentioned in the description of the natural metabolic processes, microorganisms mainly produce dihydrocoumarin from coumarin. In accordance with these processes, the present invention envisages that the biotransformation of coumarin to melilotic acid is carried out according to variant a) via o-coumaric acid, or alternatively according to variant b) via dihydrocoumarin that is formed as an intermediate. Both process steps are followed according to the invention by dehydration of the melilotic acid obtained by steps a) or b) to form dihydrocoumarin.

The dehydration is preferably carried out as a lactonization and with the aid of an acid at elevated temperatures between 30 and 200° C. and at reduced pressures of 10 to 1000 mbar. Suitable acids for the lactonization are in particular organic acids such as citric acid. Alternatively, the invention envisages that the dehydration is carried out with the aid of enzymes and preferably with the aid of esterases during which the water formed in this process should be continuously removed, which is also envisaged by the present invention.

Finally, the present invention also claims the use of the technologically produced dihydrocoumarin as a natural flavouring, and in this connection preferably in caramel, vanilla and rum flavours. However, the use of the dihydrocoumarin for the production of and/or as a component of natural, nature-identical and synthetic flavours, and preferably as honey, molasses, coconut, chocolate, brown sugar, toffee, cherry, plum, apricot, butter, condensed milk, whipped cream, marshmallow, butter beans and carob flavours, typical flavour notes of burned milk and of Graham crackers. Irrespective of whether the new dihydrocoumarin is used as a natural flavouring or together with natural, nature-identical and synthetic flavours, the present invention recommends its general use in baked good, sweets, beverages, crèmes, cereal products and milk products whereby typical products of the dietary supplement industry and functional foods are also associated.

Overall, the present invention provides a new dihydrocoumarin that is produced technologically from coumarin with the aid of a biotransformation, which in contrast to the previously known dihydrocoumarin variants fulfils the criteria of a natural product and moreover fulfils in particular the expectations of the aroma and flavouring industry.

The advantages of the present invention are illustrated by the following examples.

### EXAMPLES

### Example 1

### Biotransformation of Coumarin by Bacillus cereus

100 ml standard 1-medium (Merck VM200082; 15 g l⁻¹ peptone, 3 g l⁻¹ yeast extract, 6 g l⁻¹ NaCl, 1 g l⁻¹ glucose) was inoculated with a Bacillus cereus culture and cultured for 24 h at 30° C. and 180 rpm. Subsequently, 500 μl of a solution of coumarin in ethanol (100 mg ml⁻¹) was added and shaken further at 30° C. The concentrations of coumarin and melilotic acid after a 48 h culture period were 267 and 181 mg l⁻¹, respectively.

### Example 2

### Biotransformation of Coumarin by Pseudomonas orientalis

The procedure was similar to example 1 except that a Pseudomonas orientalis culture was used instead of Bacillus cereus. The concentrations of coumarin and melilotic acid after a 48 h culture period were 264 and 130 mg l⁻¹, respectively.

### Example 3

### Biotransformation of Coumarin by Saccharomyces cerevisiae

The procedure was similar to example 1 except that a medium optimized for yeast (20 g l⁻¹ peptone, 10 g l⁻¹ yeast extract, 20 g l⁻¹ glucose) and a Saccharomyces cerevisiae (DSMZ 2155) culture was used instead of Bacillus cereus. The concentration of melilotic acid after 144 h was 554 mg l⁻¹. Coumarin could no longer be detected.

### Example 4

### Dehydration of Melilotic Acid

1 g melilotic acid (obtained according to example 1 or 2) was heated for 1 h

to 160° C. with 10 mg citric acid in an open glass vessel. The product was examined by HPLC. It contained 90% by weight dihydrocoumarin.

1-10. (canceled)

14. A process for producing dihydrocoumarin by biotransformation, comprising contacting pure coumarin, coumarin obtained from a plant extract, or a coumarin containing plant extract, with an isolated enzyme which converts coumarin to dihydrocoumarin or a microorganism which contains an enzyme which converts coumarin to dihydrocoumarin, under conditions favoring production of dihydrocoumarin.

15. The process of claim 14, wherein said enzyme is a coumarate reductase (1.3.11), or a coumarin reductase.

16. The process of claim 14, wherein said enzyme is a Melilotus enzyme.

17. The process of claim 16, wherein said Melilotus is M. officinalis or M. alba.

18. The process of claim 14, wherein said enzyme is a Saccharomyces, Arthrobacter, Pseudomonas, Bacillus, Basidiomycetes or Fusarium enzyme.

19. The process of claim 14, wherein said microorganism is a Saccharomyces, Arthrobacter, Pseudomonas, Bacillus, Basidiomycetes or Fusarium.

20. The process of claim 14, said process comprising converting coumarin to (i) either o-coumaric acid or dihydrocoumarin, (ii) converting the product of (i) to melilotic acid, and (iii) dehydrating melilotic acid to form dihydrocoumarin and water.

21. The process of claim 20, comprising dehydrating said melilotic acid with an acid, at a temperature between 30° C. and 200° C.

22. The process of claim 21, wherein said acid is an organic acid.

23. The process of claim 22, wherein said organic acid is a citric acid.

24. The process of claim 22, comprising dehydrating melilotic acid with an enzyme.

25. The process of claim 24, wherein said enzyme is an esterase.

26. The process of claim 20, further comprising continuously removing said water.

27. The process of claim 20, comprising dehydrating melilotic acid at a pressure of from 10 to 1000 mbar.

28. The process of claim 20, wherein said microorganism is Bacillus cereus.

29. The process of claim 20, wherein said microorganism is Pseudomonas orientalis.

30. The process of claim 20, wherein said microorganism is Saccharomyces cervesial.

31. The process of claim 21, comprising dehydrating said melilotic acid with citric acid.

32. Dihydrocoumarin, biotransformed from coumarin via an isolated enzyme or a microorganism.

33. A flavouring composition comprising the dihydrocoumarin of claim 32.

34. The flavouring composition of claim 33, wherein said flavouring is caramel, vanilla or rum.

35. A method for producing a flavouring comprising adding the dihydrocoumarin of claim 32.

36. The method of claim 35, wherein said flavouring is honey, molasses, coconut, chocolate, brown sugar, toffee, cherry, plum, apricot, butter, condensed milk, whipped cream, marshmallow, butterbean, carob, burned milk, and Graham cracker.

37. A method for making a comestible selected from the group consisting of a balked good, a confection, a beverage, a cream, a cereal containing product, and a milk containing product comprising adding the dihydrocoumarin of claim 32.
